(19) United States
(12) Patent Application Publication
(10) Pub. No.: US 2010/0016290 A1
(43) Pub. Date: Jan. 21, 2010

(54) AMORPHOUS POLYMORPH OF BAZEDOXIFENE ACETATE

(76) Inventors: Livius Cotarca, Cervignano del Friuli (IT); Ivan Michieletto, Venezia (IT); Paolo Maragni, Virgilio (IT); Roberto Brescello, Abano Terme (IT)

Correspondence Address: WilmerHale/Wyeth LLC, 60 STATE STREET, BOSTON, MA, 02109, US

(21) Appl. No.: 12/369,383

(22) Filed: Feb. 11, 2009

Related U.S. Application Data
(60) Provisional application No. 61/027,725, filed on Feb. 11, 2008.

(51) Int. Cl. A61K 31/55; C07D 403/12; A61P 15/00; A61P 19/10; A61P 35/00; A61P 9/00; A61P 25/28; A61P 17/10; A61P 17/00

(52) U.S. Cl. 514/217.08; 540/602

(57) ABSTRACT

The invention provides a novel polymorphic form C of bazedoxifene acetate, methods of preparing the polymorphic form, and compositions and methods of treatment using the polymorphic form.

### RELATED APPLICATION

This application claims the benefit under 35 U.S.C. § 119(e) of U.S. Provisional Application No. 61/027,725 filed on Feb. 11, 2008, which is hereby incorporated by reference herein in its entirety.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

The invention relates to a novel amorphous polymorphic form of 1-[4-(2-azepan-1-yl-ethoxy)-benzyl]-2-(4-hydroxy-phenyl)-3-methyl-1H-indol-5-ol acetic acid (bazedoxifene acetate), methods of preparing the polymorphic form, and compositions and methods of treatment using the polymorphic form.

### BACKGROUND OF THE INVENTION

Bazedoxifene acetate (1-[4-(2-azepan-1-yl-ethoxy)-benzyl]-2-(4-hydroxy-phenyl)-3-methyl-1H-indol-5-ol acetic acid) belongs to the class of drugs typically referred to as selective estrogen receptor modulators (SERMs). Consistent with its classification, bazedoxifene demonstrates affinity for estrogen receptors (ER) but shows tissue selective estrogenic effects. Studies on bazedoxifene acetate have demonstrated that it acts as an estrogen agonist on bone and cardiovascular lipid parameters and as an estrogen antagonist on uterine and mammary tissue. Thus, bazedoxifene acetate has the potential for treating a number of different diseases or disease-like states wherein the estrogen receptor is involved.

U.S. Pat. Nos. 5,998,402 and 6,479,535 report the preparation of bazedoxifene acetate and characterize the salt as having a melting point of 174-178° C. The synthetic preparation of bazedoxifene acetate has also appeared in the general literature. See, for example, Miller et al., J. Med. Chem., 2001, 44, 1654-1657, which reports the salt as a crystalline solid having a melting point of 170.5-172.5° C. Further description of the drug's biological activity has appeared in the general literature as well (e.g. Miller, et al. Drugs of the Future, 2002, 27(2), 117-121).

International Publications WO 2005/100316 and WO 2005/100314 report the preparation of crystalline polymorph forms of bazedoxifene acetate. It is well known that the crystalline polymorph form of a particular drug is often an important determinant of the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorph form. In cases where two or more polymorph substances can be produced, it is desirable to have a method to make both polymorphs in pure form. In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc. are deemed to be critical. In other situations, a different polymorph maybe preferred for greater solubility and/or superior pharmacokinetics.

Because improved drug formulations showing, for example, better bioavailability or better stability are consistently sought, there is an ongoing need for new or purer polymorphic forms of existing drug molecules. The polymorph of bazedoxifene acetate described herein helps meet these and other needs.

### SUMMARY OF THE INVENTION

The invention provides an amorphous polymorph of bazedoxifene acetate, wherein the polymorph is a substantially pure polymorph of Form C.

The invention also provides methods for the preparation of bazedoxifene acetate polymorph Form C. In some embodiments, the methods comprise concentrating a solution of polymorph Form A under vacuum to dryness and obtaining polymorph Form C. In further embodiments, the solution of polymorph Form A is a methanol solution. In other embodiments, the methods comprise concentrating a solution of polymorph Form A under vacuum to dryness at about 0-35° C. and over a period of about 2.5 hours and obtaining polymorph Form C.

Also provided by the invention is bazedoxifene acetate polymorph Form C prepared by the methods of the invention. In some embodiments, the polymorph has a temperature of glass transition between about 68° C. and 70° C.

The invention further provides compositions comprising bazedoxifene acetate polymorph Form C and a pharmaceutically acceptable carrier. In some embodiments, the pharmaceutically acceptable carrier is suitable for oral administration and the composition comprises an oral dosage form. In some embodiments, at least about 50-99% by weight of the total of bazedoxifene acetate in the composition is present as the polymorph. In further embodiments, at least about 70%, at least about 80%, or at least about 90% by weight of the total of bazedoxifene acetate in the composition is present as the polymorph. Also provided by the invention are compositions consisting essentially of bazedoxifene acetate wherein at least about 97-99% by weight of the bazedoxifene acetate is present in the composition as the polymorph.

In other aspects, the invention provides methods of treating a disease or disorder associated with estrogen deficiency or estrogen excess, in an animal in need thereof, which comprises, administering an effective dose of an inventive composition, or a pharmaceutically acceptable salt or hydrate thereof. In some embodiments, the disease or disorder associated with estrogen deficiency or estrogen excess is selected from the group consisting of osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline, melanoma, prostate cancer, cancers of the colon, and CNS cancers.

In another aspect, the invention provides methods of treating a disease or disorder associated with proliferation or abnormal development of endometrial tissues, in an animal in need thereof, which comprises, administering an effective dose of an inventive composition, or a pharmaceutically acceptable salt or hydrate thereof. In some embodiments, the disease or disorder associated with proliferation or abnormal development of endometrial tissues is selected from the group consisting of endometrial polyps, endometriosis, and endometrial cancer.

In another aspect, the invention provides methods of lowering cholesterol, inhibiting bone loss, or treating breast cancer, in an animal in need thereof, which comprises administering an effective dose of an inventive composition, or a pharmaceutically acceptable salt or hydrate thereof. In some embodiments, the bone loss results from a disease or disorder selected from the group consisting of osteoporosis, osteopenia, osteoarthritis, hypocalcemia, hypercalcemia, Paget's disease, osteomalacia, osteohalisteresis, multiple myeloma and cancer.

In yet another aspect, the invention provides methods of treating perimenopausal, menopausal, or postmenopausal symptoms, in an animal in need thereof, which comprises administering an effective dose of an inventive composition, or a pharmaceutically acceptable salt or hydrate thereof. In some embodiments, the perimenopausal, menopausal, or postmenopausal symptom is a vasomotor disturbance, such as a hot flush.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying figures, which are incorporated in and constitute part of this specification, and together with the description, serve to explain the principles of the invention.

### BRIEF DESCRIPTION OF THE FIGURES

FIG. 1 shows a characteristic powder XRD pattern of polymorph Form C of bazedoxifene acetate.

FIG. 2 shows a characteristic IR spectrum of polymorph Form C of bazedoxifene acetate.

FIG. 3 shows a characteristic DSC thermogram of polymorph Form C of bazedoxifene acetate.

FIG. 4 shows a characteristic TGA profile of polymorph Form C of bazedoxifene acetate.

FIG. 5 shows a powder XRD pattern of Form C generated from Form A subjected to mechanical stress, as described in Example 7.

### DETAILED DESCRIPTION OF THE INVENTION

The invention provides a novel polymorph Form C of the compound 1-[4-(2-azepan-1-yl-ethoxy)-benzyl]-2-(4-hydroxy-phenyl)-3-methyl-1H-indol-5-ol acetic acid (bazedoxifene acetate), having the following formula:

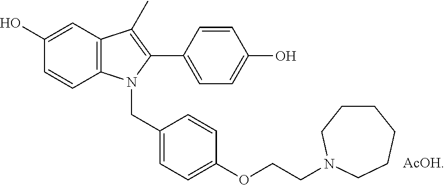

Polymorph Form C is an amorphous polymorphic form of bazedoxifene acetate. In addition to polymorph Form C of the invention, other known polymorphs of bazedoxifene acetate include crystalline polymorph Forms A and B.

As used herein, a “polymorph” refers to different crystalline forms of the same compound and includes, but is not limited to, other solid state molecular forms including hydrates and solvates of the same compound. Different polymorphs of a given compound may differ from each other with respect to one or more physical properties, such as solubility and dissociation, true density, crystal shape, compaction behavior, flow properties, and/or solid state stability. Unstable polymorphs generally convert to the more thermodynamically stable forms at a given temperature after a sufficient period of time. Metastable forms are unstable polymorphs that slowly convert to stable forms. In general, the stable form exhibits the highest melting point and the most chemical stability; however, metastable forms may also have sufficient chemical and physical stability to render them pharmaceutically acceptable. “Chemical stability” as used herein refers to the stability of certain chemical properties, including but not limited to thermal stability, light stability, and moisture stability.

The amorphous polymorph form of the invention is preferentially substantially pure, meaning the polymorph form includes less than about 15%, preferably less than about 10%, preferably less than about 5%, preferably less than about 1% by weight of impurities, including other polymorph forms of bazedoxifene acetate. In some embodiments, at least about 50-99% by weight of the total of bazedoxifene acetate in the composition is present as the amorphous polymorph. In further embodiments, at least about 70%, at least about 80%, or at least about 90% by weight of the total of bazedoxifene acetate in the composition is present as the amorphous polymorph. Also provided by the invention are compositions consisting essentially of bazedoxifene acetate wherein at least about 97-99% by weight of the bazedoxifene acetate is present in the composition as the amorphous polymorph.

The polymorph form of the invention can also be present in mixtures. In some embodiments, polymorph Form C can be present in mixtures with other polymorph Forms A and/or B. Compositions containing multiple polymorphic forms can be prepared by any suitable method, including admixture of substantially pure Forms A and B made, for example, according to any of the processes described previously in International Publications WO 2005/100316 and WO 2005/100314. Respective amounts of polymorphic forms of bazedoxifene acetate in a composition can be determined by any suitable spectroscopic method, such as X-ray powder diffraction (XRPD) or differential scanning calorimetry (DSC).

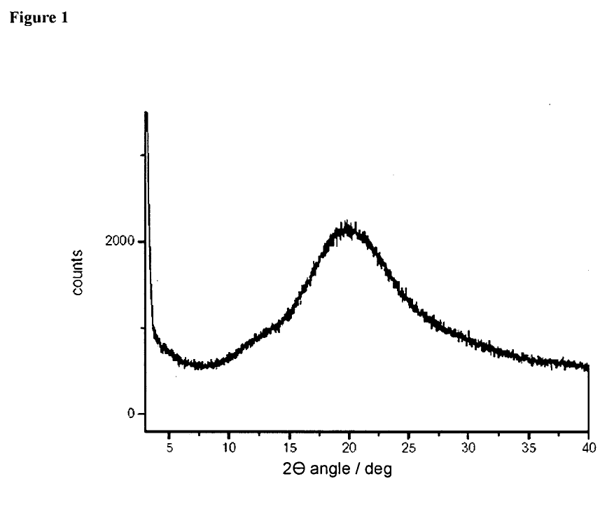

Polymorph Form C of the invention can be identified by one or more solid state analytical methods. For example, Form C can be identified by a powder X-ray diffraction pattern substantially as shown in FIG. 1. The relative intensities of the peaks can vary, depending upon the sample preparation technique, the sample mounting procedure and the particular instrument employed. Moreover, instrument variation and other factors can affect the 2-theta values.

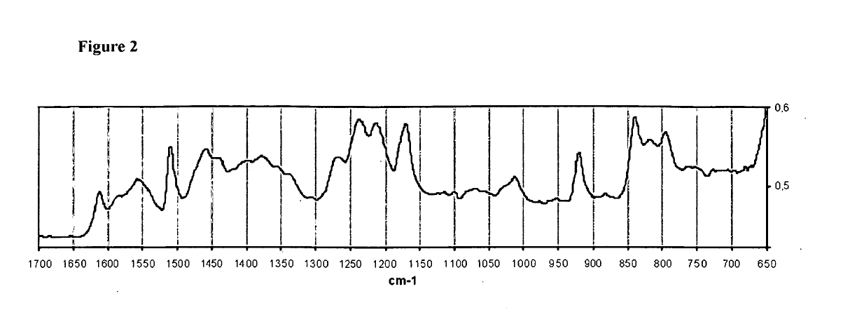

Polymorph Form C can also be identified by its characteristic infrared (IR) absorption spectrum substantially as shown in FIG. 2. In some embodiments, Form C is characterized by an infrared spectrum in KBr having one or more characteristic peaks selected from about 1213 and about 1456 cm⁻¹.

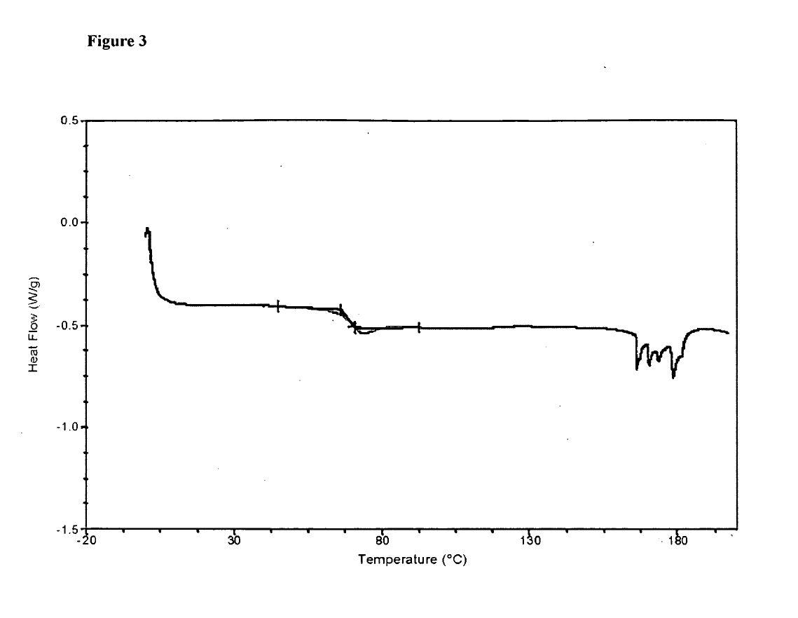

Polymorph Form C can also be identified by its characteristic DSC trace substantially as shown in FIG. 3. In some embodiments, Form C is characterized by a temperature of glass transition between about 68° C. and 70° C. Depending on the rate of heating, i.e. scan rate, at which the DSC analysis is conducted, the calibration standard used, instrument calibration, the relative humidity and upon the relative purity, the endotherms of the polymorphs may vary by about 0.01-10° C., or about 0-5° C., above or below the determined endotherms. The observed endotherm may also differ from instrument to instrument for any given sample.

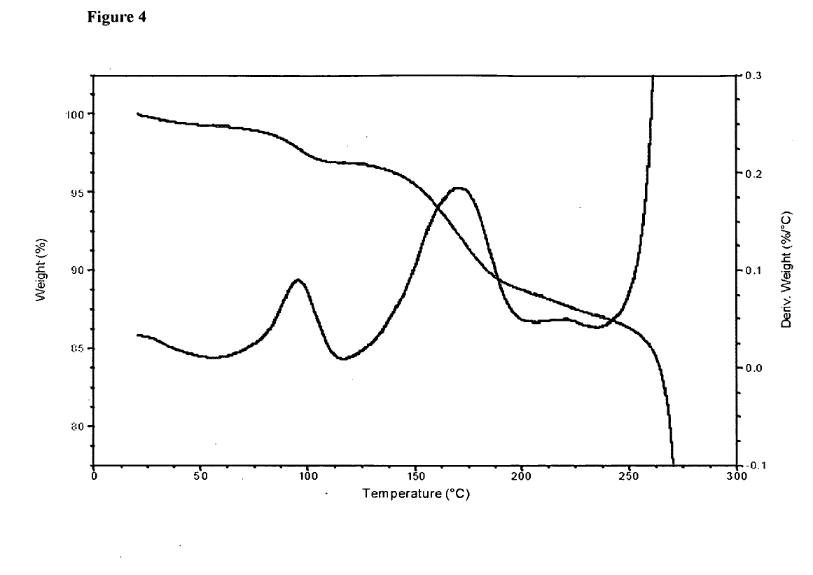

Polymorph Form C can also be identified by its characteristic thermal gravimetric analysis (TGA) profile substantially as shown in FIG. 4.

Polymorph Form C of the invention can be prepared from a solution containing a different form of the polymorph. In one embodiment, amorphous polymorph Form C can be obtained by concentrating a solution of polymorph Form A under vacuum to dryness and obtaining an amorphous polymorph Form C.

Suitable solvents for preparation of the solution of polymorph Form A from which polymorph Form C can be obtained include alcohols, including but not limited to methanol, ethanol, isopropanol, mixtures thereof, and the like. In some embodiments, the alcohol is methanol. In other embodiments, the methanol can be absolute or optionally further denatured with about 1-10% v/v toluene, about 1-10% v/v ethyl acetate, and the like. The term “methanol” as used herein includes denatured methanol.

The steps involved in the preparation of Form C can be carried out at any suitable temperature and length of time. In one embodiment, Form C can be obtained by concentrating a solution of Form A to dryness at about 25-50° C. In another embodiment, Form C can be obtained by concentrating a solution of Form A to dryness at about 35° C. In another embodiment, the concentration to dryness can occur over a period of about 2-4 hours. In another embodiment, the concentration to dryness can occur over a period of about 2.5 hours.

For purposes of administration, a polymorph of the invention may be formulated as a pharmaceutical composition. Pharmaceutical compositions of the invention comprise a polymorph and a pharmaceutically acceptable carrier, wherein the polymorph is present in the composition in an amount that is effective to treat the condition, disease or disorder of interest. The concentration of the compounds described herein in a therapeutic composition will vary depending upon a number of factors, including the dosage of the drug to be administered and the route of administration. Appropriate concentrations and dosages can be readily determined by one skilled in the art.

Pharmaceutically acceptable carriers are familiar to those skilled in the art. The compositions can be formulated as liquid solutions, and include carriers such as saline and sterile water. The compositions can also be formulated as pills, capsules, granules, or tablets which contain the polymorph along with diluents, dispersing and surface active agents, binders, and lubricants. One skilled in the art may formulate the compositions in an appropriate manner, and in accordance with accepted practices, such as those described in Remington's Pharmaceutical Sciences (Gennaro, Ed., Mack Publishing Co., Pa. 1990).

As described in U.S. Pat. No. 5,998,402, bazedoxifene and salts thereof are selective estrogen agonists with affinity for the estrogen receptor. Unlike other types of estrogen agonists, bazedoxifene and salts thereof are antiestrogenic in the uterus and can antagonize the trophic effects of estrogen agonists in uterine tissues. Accordingly, polymorphs of bazedoxifene acetate and compositions containing the same can find many uses related to treating disease states or syndromes associated with estrogen deficiency or excess of estrogen. In some embodiments, the invention provides methods of treating a disease or disorder associated with estrogen deficiency or estrogen excess. Diseases and disorders associated with estrogen deficiency or estrogen excess include osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline and other CNS disorders, as well as certain cancers including melanoma, prostate cancer, cancers of the colon, CNS cancers, among others.

Polymorphs of bazedoxifene acetate can also be used in methods of treatment for diseases or disorders which result from proliferation or abnormal development, actions or growth of endometrial or endometrial-like tissues. In some embodiments, the invention provides methods of treating a disease or disorder associated with proliferation or abnormal development of endometrial tissues. Diseases and disorders associated with proliferation or abnormal development of endometrial tissues include endometrial polyps, endometriosis, and endometrial cancer.

The polymorph of the invention can also be used in methods of inhibiting bone loss. Bone loss often results from an imbalance in an individual's formation of new bone tissues and the resorption of older tissues, leading to a net loss of bone. Such bone depletion can occur in a range of individuals, for example in post-menopausal women, women who have undergone bilateral oophorectomy, those receiving or who have received extended corticosteroid therapies, those experiencing gonadal dysgenesis, and those suffering from Cushing's syndrome. Special needs for bone, including teeth and oral bone replacement, can also be addressed using these polymorphs in individuals with bone fractures, defective bone structures, and those receiving bone-related surgeries and/or the implantation of prosthesis. In addition to the problems described above, in some embodiments the polymorph can be used in treatments for osteoporosis, osteopenia, osteoarthritis, hypocalcemia, hypercalcemia, Paget's disease, osteomalacia, osteohalisteresis, multiple myeloma and other forms of cancer having deleterious effects on bone tissues.

The polymorphic form of bazedoxifene acetate can also be used in methods of lowering cholesterol and treating breast cancer. Additionally, these polymorphs can be used for treating perimenopausal, menopausal, or postmenopausal symptoms. In some embodiments, the polymorphs can be used for contraception in pre-menopausal women, as well as hormone replacement therapy in post-menopausal women (such as for treating vasomotor disturbances such as hot flush) or in other estrogen deficiency states where estrogen supplementation would be beneficial. The polymorphs can also be used in disease states where amenorrhea is advantageous, such as leukemia, endometrial ablations, chronic renal or hepatic disease, or coagulation diseases or disorders.

The methods of the invention include systemic administration of a polymorph as disclosed herein, preferably in the form of a pharmaceutical composition. As used herein, systemic administration includes both oral and parenteral methods of administration. For oral administration, suitable compositions include powders, granules, pills, tablets and capsules as well as liquids, syrups, suspensions and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions. These methods include the step of administering, to an animal in need thereof, an effective dose of a pharmaceutical composition comprising a polymorph of bazedoxifene acetate, or a pharmaceutically acceptable salt or hydrate thereof.

The polymorphs of the invention may be synthesized according with the following non-limiting examples, which are illustrative.

### EXAMPLES

### Example 1

### Synthesis of Bazedoxifene Acetate Polymorph Form A

The starting material bazedoxifene acetate was synthesized as described in U.S. Pat. No. 5,998,402, and polymorph Form A of the bazedoxifene acetate was prepared as described in International Publication WO 2005/100316, the disclosures of which are herein incorporated by reference in their entireties.

### Example 2

### Preparation of Polymorph Form C

A suspension of bazedoxifene acetate polymorph Form A (ca. 0.4 g) in methanol (5 mL) was gently heated under stirring until a clear solution was obtained. The solution was filtered and concentrated to dryness. The solid residue was dried under vacuum at 35° C. over 2.5 hrs to give amorphous Form C (ca. 0.3 g); assay (% w/w)=98.9) as a white solid.

### Example 3

### X-Ray Powder Diffraction (XRPD)

XRPD analyses (see, e.g. FIG. 1) were carried out by means of the Philips X'Pert instrument endowed with Bragg-Brentano geometry and under the following working conditions:

X-ray tube: Copper
Radiation used: K (α1), K (α2)
Tension and current of the generator: KV 40, mA 40

### Monocromator: Graphite

Step size: 0.02
Time per step: 1.25 seconds
Starting and final angular 20 value: 3.0°-40.0°

### Example 4

### Infrared (IR) Spectroscopy

IR spectra (see, e.g. FIG. 2) were acquired by placing a few milligrams of the powder of the sample on a diamond sensor ATR (Attenuated Total Reflection) probe (Dicomp) using a React-IR 4000 system operated at 4 cm⁻¹ resolution and 128 scans between 650-4000 cm⁻¹.

Some experiments were executed by creating an ethanol vapor atmosphere around the ATR sensor at room temperature. Polymorph Form C placed on the ATR sensor and maintained in the presence of ethanol vapor was found to transform rapidly into polymorph Form B. After two hours, only polymorph Form B was present.

### Example 5

### Differential Scanning Calorimetry (DSC)

DSC measurements (see, e.g. FIG. 3) were carried out in crimped aluminum pans at a scan rate of 10° C./min from 0° C. to 200° C. under nitrogen purge using a TA Instruments Q100 calorimeter.

### Example 6

### Thermal Gravimetric Analysis (TGA)

TGA analyses were carried out with platinum pans at a scan rate of 4° C./min from 25° C. to 280° C. using a TA Instruments Q500 thermogravimeter.

### Example 7

### Generation of Polymorph Form C from Processing of Polymorph Form A

Mortar and pestle trials were conducted to test the behavior of polymorph Form A under mechanical stress and to test and measure the kinetics of conversion of crystalline Form A to Form B under mechanical stress (possibly via amorphous Form C).

The experimental protocol was as follows: each mix of 0.5 g sample of bazedoxifene acetate Form A was diluted with 0.5 mL of reaction solvent, seeded with Form B or amorphous Form C, and then grinded with a mortar and pestle for 1 or 10 minutes, thoroughly mixing the different solid forms; the obtained mix was scraped off the mortar and pestle using a spatula; and the ground polymorph mix was placed in a vial and dried at 50° C. under vacuum. All the recovered samples were submitted to DSC, TGA, XRPD, and NMR analysis.

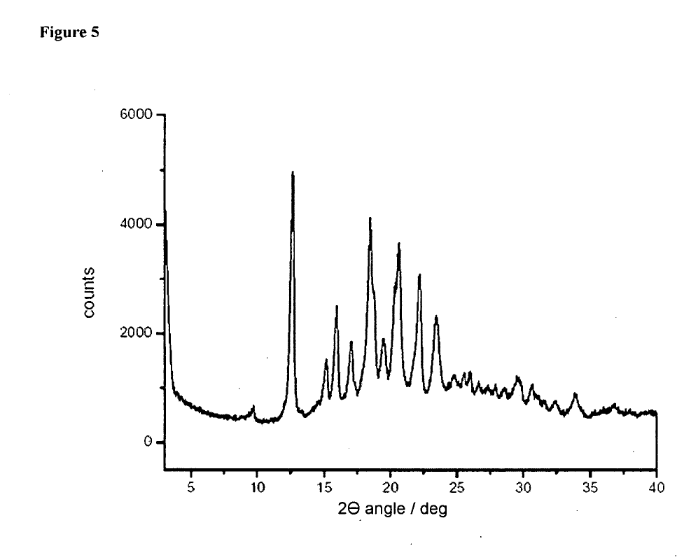

The results from DSC analysis of the nine samples obtained are shown in Table 1. As measured by DSC analysis, sample 8 shows a clear increase in the level of polymorph Form B accompanied by a significant amount of amorphous Form C. This result was confirmed by the powder X-Ray diffractogram of sample 8 shown in FIG. 5 (FIG. 5 does not clearly show the representative peaks for polymorph Form B).

TABLE 1

| | | | | % w/w | |
|---|---|---|---|---|---|
| | | | Seed | Form B by | |
| Sample | Solid | Solvent | (notes) | DSC** | Notes |
| 1 | Form A | — | — | n.d. | |
| 2 | Form A | React. | — | n.d. | |
| | | solv.* | | | |
| 3 | Form A | water | — | n.d. | |
| 4 | Form A | React. | 0.01 < Form | 0.5*** | |
| | | solv.* | B < 0.1% w/w | | |
| 5 | Form A | React. | Form B = 0.5% | 1*** | |
| | | solv.* | w/w | | |
| 6 | Form A | React. | — | 0.3 | |
| | (0.1% | solv.* | 1 min mixing | | |
| | Form B) | | | | |
| 7 | Form A | React. | — | 0.1 | About 30% |
| | (0.1% | solv.* | 10 min mixing | [symbol] | decrease in |
| | Form B) | | | | the Enthalpy |
| | | | | | of Fusion |
| | | | | | (Form A) |
| 8 | Sample 5 | React. | — | 14 | About 50% |
| | | solv.* | 10 min mixing | [symbol] | decrease in |
| | | | | | the Enthalpy |
| | | | | | of Fusion |
| | | | | | (Form A) |
| 9 | Form A | — | AM = 1% w/w | n.d. | |
| | | | 10 min mixing | | |
| | | | [symbol] | | |

AM: Amorphous Form (ca. 5% of crystalline material): reference sample
DSC data: flex at ca. 70-72° C. (glass transition temperature, Tg)
*Reaction solvents (vol ratio): EtOAc/EtOH (den)/Toluene/Water = 0.20/0.75/0.04/0.01
**DSC analysis:
Method: 2-3 mg sample; 5° C./min; 150->190° C.; all samples were measured in crimped aluminum pans
Instrument: TA Q100 “R-DSC-01-A”
The % w/w amount of Form B was estimated considering an Enthalpy of Fusion for Form B = 115J/g (average value in duplicate).
One further DSC thermogram was measured for each sample in the temperature range of −20-200° C. (10° C./min).
The DSC thermogram measured in the temperature range of −20-200° C. (10° C./min) for the first five sample trials did not show any significant variation.
***Quantitative evaluation of Form B was difficult because of an unusual loss of resolution. [symbol]
The DSC thermogram did not clearly show a glass transition (tg) as for the reference sample. [symbol]
Difficult integration due to a poor resolution. The DSC thermogram did not clearly show a glass transition (tg) as for the reference sample. [symbol]
The DSC thermogram did not show any significant amount of amorphous compound in the sample.

### Example 8

### Stability/Conversion of Polymorph Form C

Trials were conducted to check for the stability/conversion of amorphous material into crystalline form, including the possible conversion of amorphous Form C into one crystalline form (e.g., Form B) during the storage of bazedoxifene acetate in an organic solvent atmosphere.

In the first set of experiments, four vials each containing samples (300 mg) of amorphous solid were seeded with Form A or Form B in a closed system saturated with an organic solvent (crystallization mixture). The crystallization mixture was prepared by mixing: EtOH (den)/EtOAc/demiWater/ascorbic acid/acetic acid/toluene=123.7 g/37.7 g/0.502 g/0.199 g/1.17 g/7.17 g. The mixture was placed into a TLC chamber (d=11 cm; h=11.5 cm) followed by the vials containing amorphous material. The TLC chamber was then closed down. After 24 hrs at room temperature, the solid samples were dried at 35° C. under vacuum over 20 hrs and then submitted to DSC, TGA and FTIR analysis.

A summary of data collected from DSC and FTIR analysis is shown in Table 2 and shows the complete conversion of all amorphous samples into crystalline Form B.

TABLE 2

| | | | Amount & type |
|---|---|---|---|
| | | T glass | of crystalline* |
| Sample | Sample Description | (° C.) | (% w/w) |
| A | Amorphous | n.d. | 100 (Form B)** |
| B | Amorphous + 1% Form B | n.d. | 100 (Form B) |
| C | Amorphous + 1% Form A | n.d. | 100 (Form B) |
| D | Amorphous containing | n.d. | 100 (Form B) |
| | 20% of crystalline | | |

*All DSC thermograms showed an Enthalpy of Fusion lower than the typical value for STD Form B (115 J/g) and did not show any significant amount of residual amorphous compound (Tg = 68-70° C.).
**Polymorph type was assigned also by comparison of the FTIR spectra recorded for the STD Form A and B.

A summary of data collected from TGA analysis is shown in Table 3. TGA analysis did not show evidence for the formation of solvates.

TABLE 3

| | % weight loss |
|---|---|
| Sample | (100->230° C.) * |
| A | 12.6 ** |
| B | 11.6 ** |
| C | 11.6 ** |
| D | 12.5 |

* Range of temperature in which the loss of acetic acid is typically observed.
** About 0.1% weight loss is observed in the range of temperature 70->100° C.

In the second set of experiments, the conversion of amorphous material into crystalline (Form B) was monitored over a more restricted time frame. Four vials each containing samples (160 mg) of amorphous solid were left in a closed system saturated with organic solvent (crystallization mixture) at room temperature and analyzed by DSC, TGA and FTIR after standing in the saturated system for 2, 3.5, and 6 hours, respectively. Each sample was analyzed both just as it was and after drying at 40° C. under vacuum.

A summary of data collected from DSC and FTIR analysis is shown in Table 4 and shows the complete conversion of the amorphous form into the crystalline Form B from the sample kept 3.5 hrs in a closed system saturated with organic solvent (crystallization mixture) at room temperature.

TABLE 4

| | | | |
|---|---|---|---|
| | | T glass | Amount & type of |
| Sample * | Time | (° C.) | crystalline** (% w/w) |

| | | | | |
|---|---|---|---|---|
| A | 2 | hrs [symbol] | 69.7 | ~50 (Form B) |
| B | 3.5 | hrs [symbol] | n.d. | ~80 (Form B) |
| C | 6 | hrs | n.d. | 100 (Form B) |
| D | 8 | hrs | n.d. | 100 (Form B) |

* No difference detected for each sample analyzed just as it was and after drying at 40° C. under vacuum.
** Estimated value considering the Enthalpy of Fusion recorded in the area of thermogram typical for Form A and B. [symbol]
The sample showed an exothermal peak between 98 and 150° C.

A summary of data collected from TGA analysis is shown in Table 5. The results for samples A and B suggest the formation of a possible intermediate solvate form (with ethanol) during the transformation from amorphous state to crystalline Form B. This assumption was also confirmed by FT-IR analyses on the samples.

TABLE 5

| | | | % weight loss | % weight loss |
|---|---|---|---|---|
| Sample * | Time | | (60->110° C.) | (110->240° C.) |
| A | 2 | hrs | 2.3 | 12.1 |
| B | 3.5 | hrs | 2.1 | 12.1 |
| C | 6 | hrs | — | 12.9 |
| D | 8 | hrs | — | 13.0 |

* All samples analyzed after drying at 40° C. under vacuum.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

We claim:

1. An amorphous polymorph of bazedoxifene acetate, wherein the polymorph is a substantially pure polymorph of Form C.

2. A method for the preparation of the amorphous polymorph of claim 1, which comprises concentrating a solution of polymorph Form A under vacuum to dryness and obtaining the amorphous polymorph of claim 1.

3. The method of claim 2, wherein the solution of polymorph Form A is a solution in an alcohol.

4. The method of claim 3, wherein the alcohol is methanol.

5. The method of claim 2, wherein the solution of polymorph Form A is concentrated to dryness at about 25-50° C.

6. The method of claim 5, wherein the solution of polymorph Form A is concentrated to dryness at about 35° C.

7. The method of claim 2, wherein the solution of polymorph Form A is concentrated to dryness over about 2.5 hours.

8. The polymorph Form C of bazedoxifene acetate prepared by any of the methods of claims 2 to 7.

9. The amorphous polymorph of claim 1, wherein the polymorph has a temperature of glass transition between about 68° C. and 70° C.

10. The amorphous polymorph of claim 1 with a powder X-ray diffraction pattern substantially as shown in FIG. 1.

11. The amorphous polymorph of claim 1 with an infrared spectrum substantially as shown in FIG. 2.

12. The amorphous polymorph of claim 1 with an infrared spectrum comprising one or more characteristic peaks selected from about 1213 and 1456 cm⁻¹.

13. The amorphous polymorph of claim 1 with a differential scanning calorimetry trace substantially as shown in FIG. 3.

14. The amorphous polymorph of claim 1 with a thermal gravimetric analysis profile substantially as shown in FIG. 4.

15. A composition comprising the amorphous polymorph of claim 1, or a pharmaceutically acceptable salt or hydrate thereof, and a pharmaceutically acceptable carrier.

16. The composition of claim 15, wherein the pharmaceutically acceptable carrier is suitable for oral administration and the composition comprises an oral dosage form.

17. The composition of claim 15, wherein at least about 50-99% by weight of the total of bazedoxifene acetate in said composition is present as said polymorph.

18. The composition of claim 15, wherein at least about 70% by weight of the total of bazedoxifene acetate in said composition is present as said polymorph.

19. The composition of claim 15, wherein at least about 80% by weight of the total of bazedoxifene acetate in said composition is present as said polymorph.

20. The composition of claim 15, wherein at least about 90% by weight of the total of bazedoxifene acetate in said composition is present as said polymorph.

21. A composition consisting essentially of bazedoxifene acetate wherein at least about 97-99% by weight of said bazedoxifene acetate is present in said composition as the polymorph of claim 1.

22. A method of treating a disease or disorder associated with estrogen deficiency or estrogen excess, in an animal in need thereof, comprising administering an effective dose of the composition of claim 15.

23. The method of claim 22, wherein the disease or disorder associated with estrogen deficiency or estrogen excess is selected from the group consisting of osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline, melanoma, prostate cancer, cancers of the colon, and CNS cancers.

24. A method of treating a disease or disorder associated with proliferation or abnormal development of endometrial tissues, in an animal in need thereof, comprising administering an effective dose of the composition of claim 15.

25. The method of claim 24, wherein the disease or disorder associated with proliferation or abnormal development of endometrial tissues is selected from the group consisting of endometrial polyps, endometriosis, and endometrial cancer.

26. A method of lowering cholesterol, in an animal in need thereof, comprising administering an effective dose of the composition of claim 15.

27. A method of inhibiting bone loss, in an animal in need thereof, comprising administering an effective dose of the composition of claim 15.

28. The method of claim 27, wherein the bone loss results from a disease or disorder selected from the group consisting of osteoporosis, osteopenia, osteoarthritis, hypocalcemia, hypercalcemia, Paget's disease, osteomalacia, osteohalisteresis, multiple myeloma and cancer.

29. A method of treating breast cancer, in an animal in need thereof, comprising administering an effective dose of the composition of claim 15.

30. A method of treating perimenopausal, menopausal, or postmenopausal symptoms, in an animal in need thereof, comprising administering an effective dose of the composition of claim 15.

31. The method of claim 30, wherein the perimenopausal, menopausal, or postmenopausal symptom is a vasomotor disturbance.

32. The method of claim 31, wherein the vasomotor disturbance is a hot flush.
